(19) United States
(12) Patent Application Publication
(10) Pub. No.: US 2008/0214939 A1
(43) Pub. Date: Sep. 4, 2008

(54) PROBE FOR TRANSESOPHAGEAL ECHOCARDIOGRAPHY WITH ERGONOMIC CONTROLS

(76) Inventor: Edward Paul Harhen, Duxbury, MA (US)

Correspondence Address: PROSKAUER ROSE LLP;PATENT DEPARTMENT, 1585 BROADWAY, NEW YORK, NY, 10036-8299, US

(21) Appl. No.: 12/018,381

(22) Filed: Jan. 23, 2008

Related U.S. Application Data
(60) Provisional application No. 60/886,478, filed on Jan. 24, 2007.

(51) Int. Cl. A61B 8/13

(52) U.S. Cl. 600/462; 16/422

(57) ABSTRACT

A connectorized ultrasound probe includes a distal section that is configured for insertion into a patient's body and a proximal section configured to interface the distal section with an ultrasound system. The distal section is easily attachable and detachable from the proximal section using at least one set of connectors. When connected, a user-operated actuator located on the proximal section controls the bending of the distal section, and the ultrasound system sends driving signals to and receives return signals from the ultrasound transducer via the proximal section. The proximal section also has a control bottom ergonomically arranged in relation to the actuator to allow actuation of the actuator and button with one hand.

### CROSS REFERENCE TO RELATED APPLICATIONS

This application claims the benefit of U.S. provisional application No. 60/886,478, filed Jan. 24, 2007, which is incorporated herein by reference.

### BACKGROUND

U.S. application Ser. No. 10/996,816, filed Nov. 24, 2004, which is incorporated herein by reference, describes a unique ultrasound probe, transducer, and associated algorithm. The probe disclosed in the '816 application is significantly narrower than prior art devices, and can be left in place for extended periods of time. The primary intended use of that probe is for monitoring of the heart using echocardiography. FIG. 1 is a schematic representation of that probe 100. The probe has a flexible shaft 112 affixed to the end of an endoscope style control handle 104, and the distal end 116 of the probe 100 contains the ultrasound transducer 118. To use the probe, the distal end 116 is manipulated into position in the esophagus, and a bending mechanism is then actuated using actuator 102, which causes the bending section 114 of the probe to bend. In the context of echocardiography, this bending action is used to position the ultrasound transducer 118 in the fundus of the stomach to obtain an image of the transgastric short axis view of the heart. The handle 104 is connected to a connector 42 on the ultrasound system 40 via a cable 106 that terminates at a connector 108.

In the setting of an intensive care unit (ICU), patients are often maintained in a quiescent state for both the well-being of the patient and to facilitate the monitoring of various physiological functions. Leaving the probe 100 in place for extended periods of time, however, can create difficulties in common situations when the patient must be moved. (Examples of such situations include moving the patient to clean him or her, to prevent pressure sores, or to perform routine procedures.) If the probe 100 is kept in the patient while the probe is hooked up to the ultrasound system 40, moving the patient could be extremely difficult.

One solution to this problem is to detach the probe 100 from the ultrasound system 40 by disconnecting the probe's connector 108 from the ultrasound system's connector 42 before the patient is moved, to leave those portions of the probe that remain outside the patient's body 102-108 resting on a tray or a hook. However, since the handle 104 and associated cable portions 106 of the transesophageal echo (TEE) probe that remain attached to the patient are relatively large and heavy, this solution is somewhat clumsy, and requires an extra degree of awareness from the attendants so as to not dislodge the device or cause other problems due to paying too much attention to the device.

### SUMMARY

A connectorized ultrasound probe includes a distal section that is configured for insertion into a patient's body and a proximal section configured to interface the distal section with an ultrasound system. The distal section is easily attachable and detachable from the proximal section using at least one set of connectors. When connected, a user-operated actuator located on the proximal section controls the bending of the distal section, and the ultrasound system sends driving signals to and receives return signals from the ultrasound transducer via the proximal section. The proximal section also has a control bottom ergonomically arranged in relation to the actuator to allow actuation of the actuator and button with one hand.

### BRIEF DESCRIPTION OF THE DRAWINGS

FIG. 1 is a schematic representation of the transesophageal echocardiography ultrasound probe disclosed in the '816 application.

FIG. 2 is a schematic representation of a first embodiment of an improved ultrasound probe for transesophageal echocardiography in accordance with the present invention.

FIG. 3 is an isometric view of an implementation of the ultrasound probe of FIG. 2, with a transducer assembly connected to a matching actuator assembly.

FIG. 4 is a first detailed view of the interface between the transducer assembly and the actuator assembly of the FIG. 3 embodiment.

FIG. 5 is a detailed view of the interface portion of the actuator assembly of the FIG. 3 embodiment.

FIG. 6 is a detailed view of the interface portion of the transducer assembly of the FIG. 3 embodiment.

FIG. 7 shows the internal components of the transducer assembly of FIG. 6, with the lid removed.

FIG. 8 shows the transducer assembly of FIG. 6, with certain components removed to make the lower components visible.

FIG. 9 shows the electrical and mechanical interactions between the transducer assembly and the actuator assembly when those two assemblies are mated together.

FIG. 10 is another embodiment of an improved ultrasound probe for transesophageal echocardiography in accordance with the present invention.

FIG. 11 is a detail of the mechanical connection on the actuator assembly side of the probe of FIG. 10.

FIG. 12 is a detail of the mechanical connection on the transducer assembly side of the probe of FIG. 10.

FIG. 13 is an alternative preferred variation of the FIG. 3 embodiment.

FIG. 14 is another alternative preferred variation of the FIG. 3 embodiment.

### DETAILED DESCRIPTION OF THE PREFERRED EMBODIMENTS

The drawbacks associated with a large handle and cabling that remains connected to the patient while the probe is in the patient's esophagus can be avoided or minimized by using a connectorized probe, with a distal portion that remains installed in the patient, and a detachable handle portion that interfaces with the distal portion. The connector passes both mechanical and electrical signals between the two portions. Optionally, the distal portion may be disposable, in which case it is preferable to reduce the cost of the distal portion. Because it is not disposable, the cost of the handle portion is less critical.

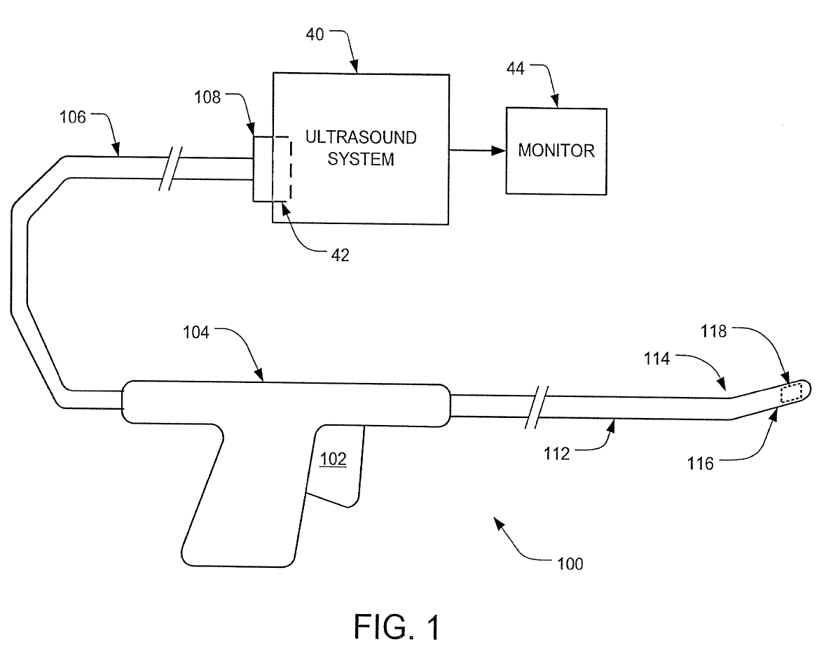

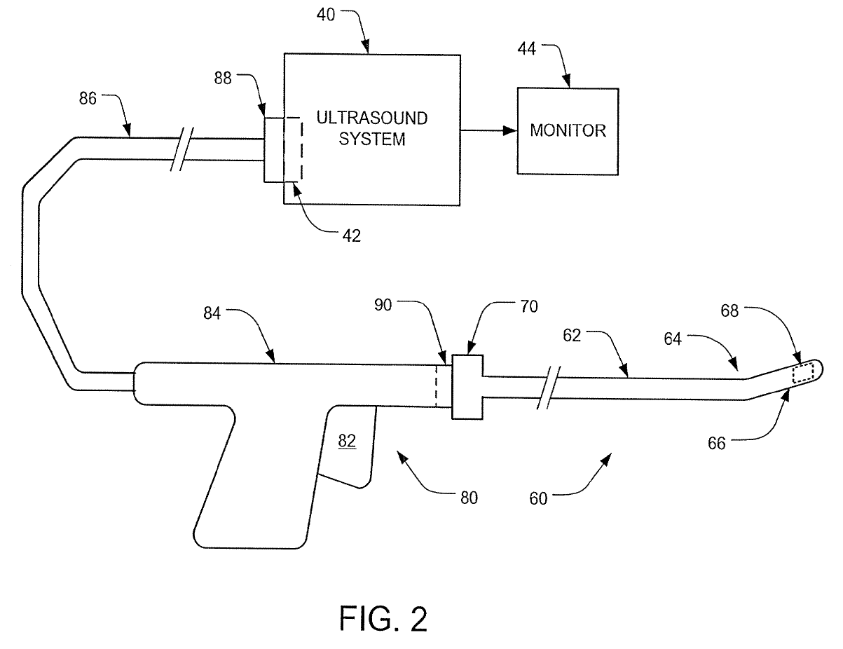

FIG. 2 is a schematic representation of an embodiment of the invention, with a probe that includes an actuator assembly 80 and a transducer assembly 60. The actuator assembly 80 includes a control handle 84 with an actuator 82. The handle 84 is connected to a connector 42 on the ultrasound system 40 via a cable 86 that terminates at a connector 88. The transducer assembly 60 has a flexible shaft 62 affixed to the end of a connector 70, and the distal end 66 of the probe contains the ultrasound transducer 68. To use the probe, the actuator assembly 80 and the transducer assembly 60 are connected together by mating the first connector 90 with the second connector 70. The distal end 68 is then manipulated into position in the esophagus. The transducer assembly 60 includes a bending mechanism that is actuatable by the actuator 82 when the actuator assembly 80 and the transducer assembly 60 are connected together. This causes the bending section 64 of the probe to bend to provide an end result that is similar to the bending achieved in the unitary probe described above in connection with FIG. 1.

Now, when it becomes necessary to move the patient, the transducer assembly 60 is disconnected from the actuator assembly 80 at the connectors 70, 90, so that the only parts that remain protruding from the patient will be the proximal end of the shaft 62 and the connector 70. Since those portions are relatively small and light compared to the handle 104 and cable 106 of the probe 100 depicted in FIG. 1, it becomes much easier to leave the distal end of the probe in the patient when the patient has to be moved or cared for.

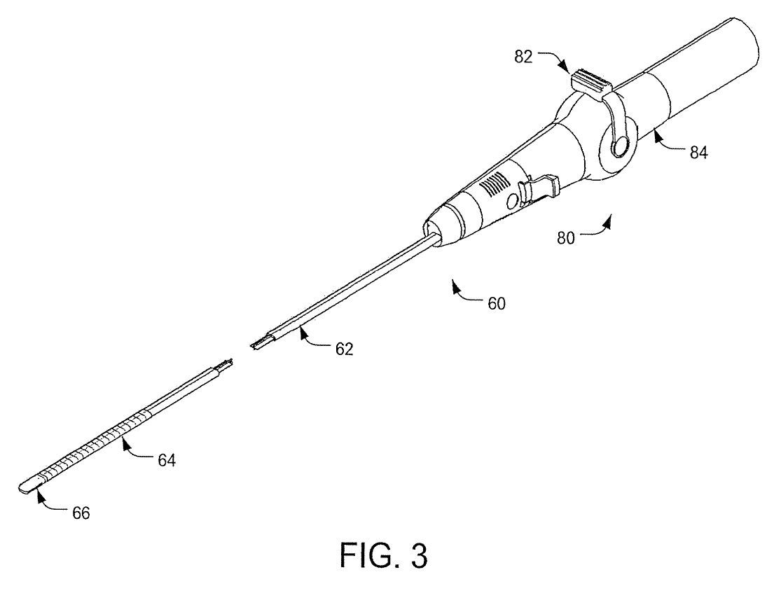

FIG. 3 depicts a preferred implementation of the FIG. 2 embodiment, with the transducer assembly 60 mounted to the actuator assembly 80. The transducer assembly 60 includes a flexible shaft 62 (shown with a break to denote its long length) that has a bending section 64. The shaft 62 is preferably less than 6 mm in diameter, and preferably on the order of 1 m in length for an adult version of the device. Those dimensions may be scaled down appropriately for pediatric and neonatal patients. The distal end 66 of the transducer assembly 60 houses the ultrasound transducer which is preferably transversely oriented with respect to the proximal distal axis. In alternative embodiments, other transducer configurations may be used in place of the transversely oriented transducer (e.g., a two-dimensional ultrasound transducer or a rotating multi-plane transducer). The actuator assembly 80 includes a handle 84 with a user-operated actuator 82 mounted on the handle. A cable 86 with a connector 88 at its proximal end (both shown in FIG. 2) extends from the proximal end of the handle 84. This connector 88 mates with a corresponding connector 42 on the ultrasound system 40 (all shown in FIG. 2).

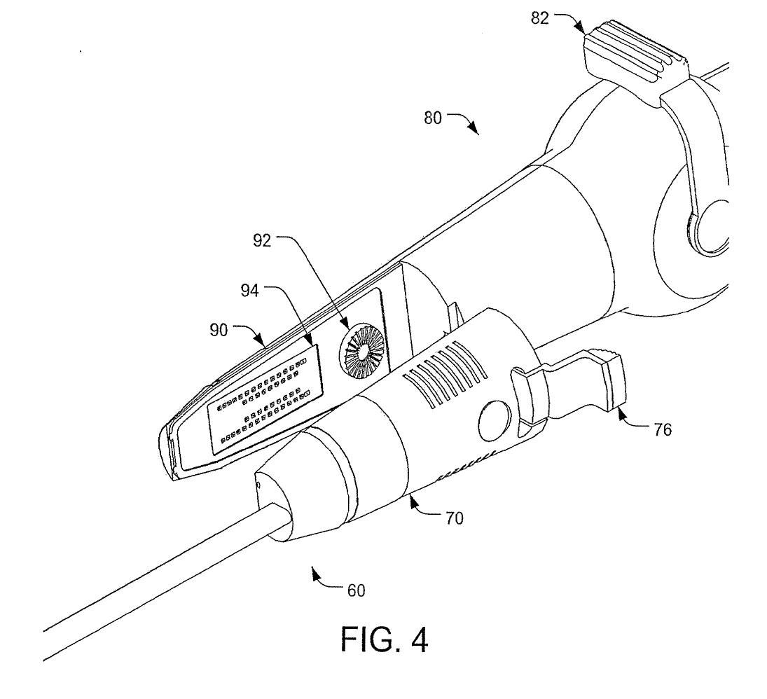

FIG. 4 is an exploded detail view of the interface between the actuator assembly 80 and the transducer assembly 60. The actuator assembly 80 includes a first connector 90 that interfaces with the transducer assembly 60, and the transducer assembly 60 includes a second connector 70 that interfaces with the actuator assembly 80. The first connector 90 includes a first electrical interface 94, which is used to make electrical connect with a mating connector (not shown) on the second connector 70. In the illustrated embodiment, the first electrical interface 94 comprises a series of conductive pads, which are preferably gold plated. The pads may be flat or raised. Preferably, the first connector is constructed to be watertight so that the first connector can be immersed in a liquid sterilant (e.g., Cidex glutaraldehyde, peroxide sterilants, etc.), and using simple, stationary pads helps achieve the desired watertightness, which facilitates re-use of the actuator assembly 80 for multiple patients. When the second connector 70 is mated to the first connector 90, corresponding contacts on the second connector 70 line up with the contacts of the first electrical interface 94 so that electrical signals can pass between the actuator assembly 80 and the transducer assembly 60.

The ultrasound system 40 communicates with the ultrasound transducer 68 (both shown in FIG. 2) by sending and receiving appropriate signals into the actuator assembly 80 via the connector 42, the connector 88, and the cable 86 (all shown in FIG. 2). The signals that travel through the cable 86 are routed to the first electrical interface 94 on the first connector 90 e.g., by running appropriately shielded wires from the distal end of the cable 86 directly to the first electrical interface 94. Optionally, appropriate intervening circuitry (e.g., amplifiers, signal conditioners, etc.) may be interposed between the first electrical interface 94 and the cable 86. The remainder of the path to the transducer is described below in connection with the transducer assembly 60.

The first connector 90 also includes an output actuator 92 that is designed to mate with a corresponding member on the second connector 70 when the second connector 70 is connected to the first connector 90. The output actuator 92 is linked to the user-operated actuator 82 by an appropriate mechanism such that the output actuator moves in response to user actuation of the user-operated actuator 82. The link between the user-operated actuator 82 and the output actuator 92 may be implemented using any of a variety of conventional techniques, including but not limited to gears, pull wires, servo motors, stepper motors, hydraulics, as well as numerous other techniques that will be apparent to persons skilled in the relevant arts. The output actuator 92 and the user-operated actuator 82 are preferably also made using a watertight construction (e.g., using O rings or other sealing techniques) to facilitate liquid sterilization of the actuator assembly 80.

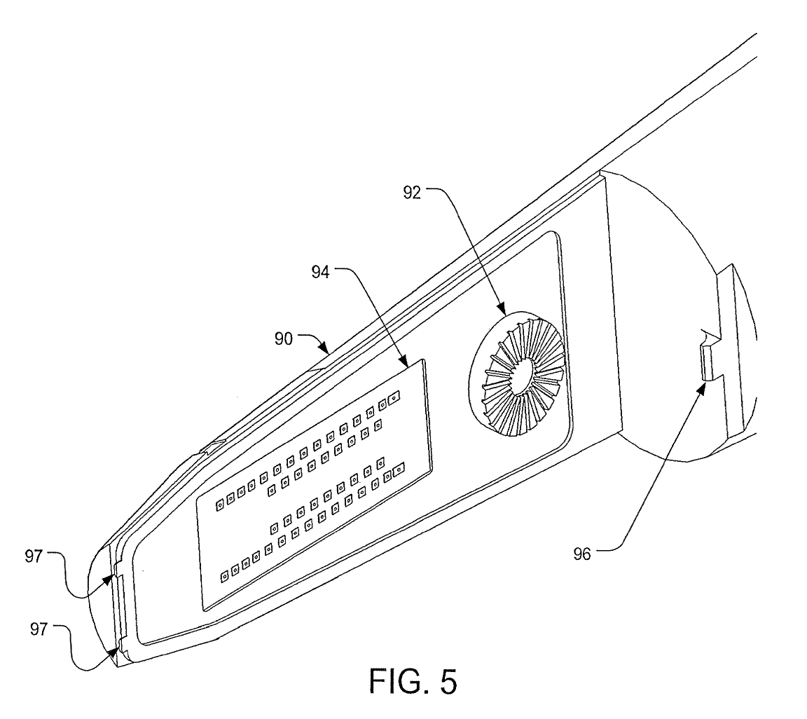

FIG. 5 shows the first connector 90 in even greater detail. As explained above, the output actuator 92 rotates in response to actuations of the user-operated actuator 82. The surface of the output actuator 92 is preferably made of a material that will have a high coefficient of friction when it is pressed against a corresponding member in the second connector 70. Examples of suitable materials for the output actuator include rubber, polyethylene, polystyrene, vinyl, etc. Optionally, a plurality of radial grooves may be cut into the surface of the output actuator 92 to help the output actuator 92 better “grab” the corresponding surface on the second connector 70.

As best seen in this view, the first connector 90 includes a number of mounting members for latching the first connector onto the second connector. Although the illustrated embodiment depict mounting members in the form of a pair of small tabs 97 at the distal end and a larger tab 96, persons skilled in relevant arts will recognize that any of a wide variety of conventional latching mechanism may be used.

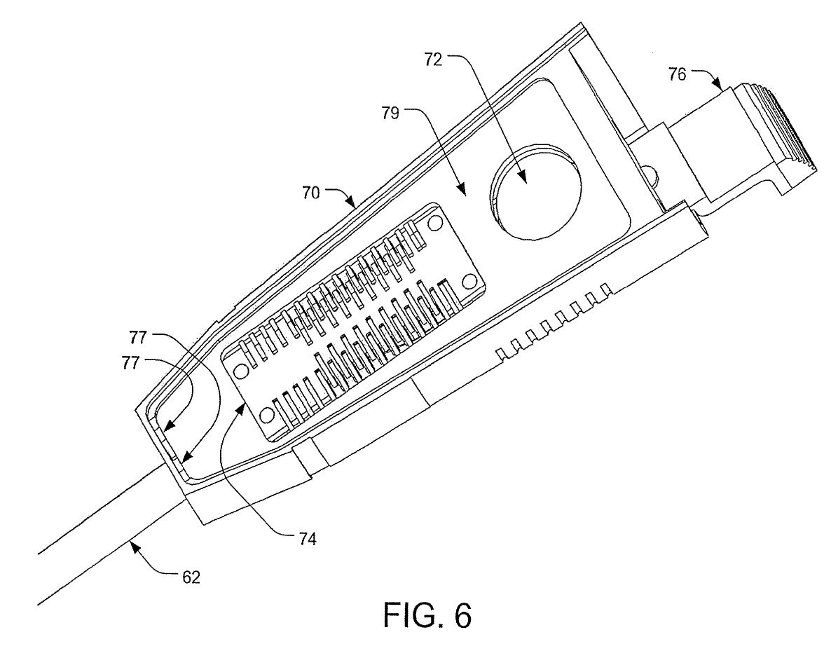

FIG. 6 is a front view of the second connector 70. The second connector 70 is configured to mate with the first connector 90. To do this, the second connector 70 contains a second electrical interface 74 that lines up the first electrical interface 94 of the first connector 90. In the illustrated embodiment, the second electrical interface 74 is made using a plurality of spring loaded fingers positioned so that, when the second connector 70 is connected to the first connector 90, the fingers of the second electrical interface 74 will line up with the pads of the first electrical interface 94 (shown in FIGS. 4, 5). The second connector 70 also contains a control actuator 72 that lines up the output actuator 92 of the first connector 90, so that the output actuator 92 can drive the control actuator 72. In the illustrated embodiment, the control actuator 72 is a rotating wheel that is designed to be driven by rotation of the output actuator 92. Of course, a wide variety of alternative arrangements for actuating alternative control actuators will be readily apparent to persons skilled in the relevant arts. Note that when the transducer assembly 60 is disposable and will be discarded after each use, it is not necessary to make the second connector 70 watertight.

To connect the first and second connectors, the second connector 70 is attached to the first connector 90 by aligning the notches 77 of the second connector 70 with tabs 97 of the first connector 90, then squeezing the proximal end of second connector 70 towards the first connector 90. The latching arm 76 on the second connector 70 is designed to snap into position on the first connector by interacting with tab 96 (shown in FIG. 5). When the first connector 70 is attached to the first connector 90 in this manner, the second electrical interface 74 of the second connector 70 makes electrical connect with the first electrical interface 94 of the first connector 90, so that electrical signals can travel back and forth between the first electrical interface 94 and the second electrical interface 74. In addition, the control actuator 72 makes mechanical connect with the output actuator 92 of the first connector 90, so that when the output actuator 92 is rotated in response to operation of the user operated actuator 82 (shown in FIG. 4) the control actuator 72 will be driven by the output actuator 92 and followed the rotation of the output actuator 92. A lid 79 protects the internal components of the second connector 70 from damage, and has cutouts to provide access to the second electrical interface 74 and the control actuator 72. Note that while FIGS. 4-9 depict first and second electrical interfaces 94, 74 using pads and fingers designed to contact the pads, numerous alternative electrical interfaces (e.g., pins and mating sockets) may be substituted therefore, as will be appreciated by persons skilled in the relevant arts.

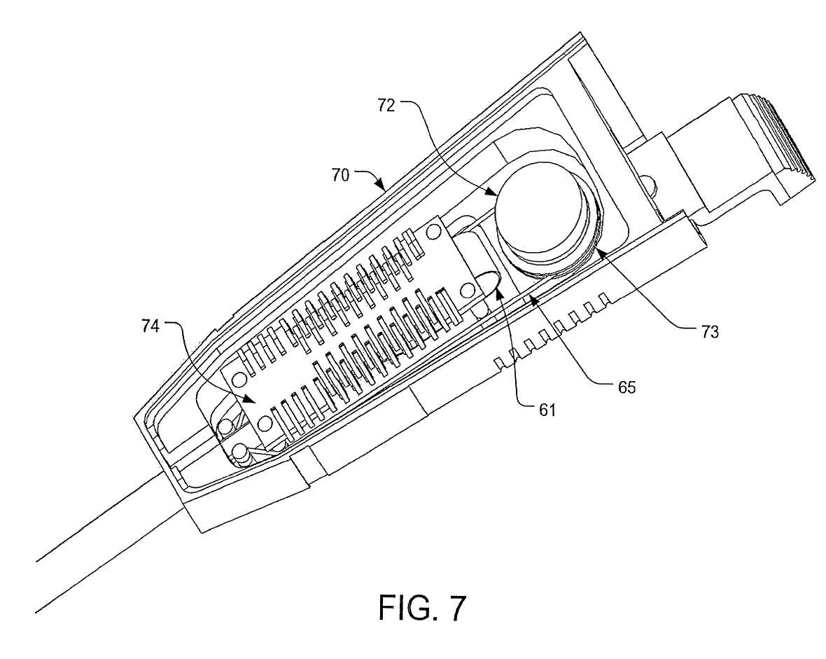

FIG. 7 is another view of the second connector 70 shown in FIG. 6, with the lid 79 removed. This view reveals that the rotating control actuator 72 is attached to a pulley 73 that causes the pull wires 65 to move when the control actuator 72 is rotated. This view also shows a portion of the ribbon cable 61, which is the wiring that connects the second electrical interface 74 to the transducer 68 (shown in FIG. 2) at the distal end 66 of the transducer assembly 60. Preferably, a ground plane is provided on both sides of the ribbon cable. In less preferred embodiments one or both of those ground planes may be omitted, or wiring configurations other than ribbon cable may be used. Optionally, appropriate intervening circuitry (e.g., amplifiers, signal conditioners, etc.) may be interposed between the second electrical interface 74 and the transducer 68.

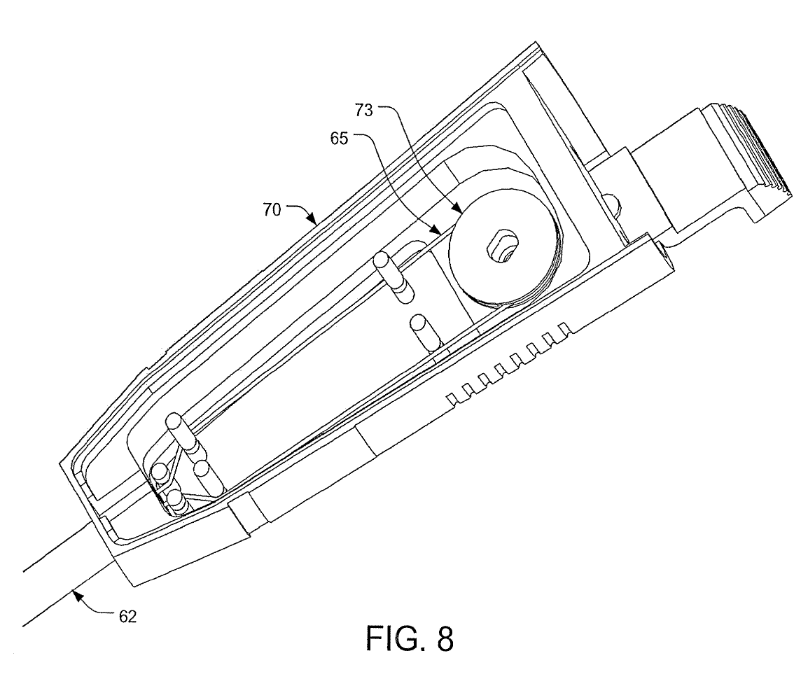

FIG. 8 shows yet another view of the second connector 70 of FIGS. 6 and 7, but with the lid 79, the second electrical interface 74, the wiring 61, the control actuator 72, and the pulley's axle all removed to show the lower components of the second connector 70. This view more clearly shows how the pulley 73 moves the pull wires 65, which extend out distally through the shaft 62. When the pull wires 65 move (in response to rotation of the pulley), the pull wires operate the bending section 64 (shown in FIG. 3) in any conventional manner. Since the pull wires 65 cause the bending section 64 to bend, and the pull wires 65 are moved by rotation of the pulley 73, and rotation of the pulley 73 occurs in response to rotation of the control actuator 72 (shown in FIGS. 6 and 7), the net result is that rotation of the control actuator 72 causes the bending section 64 to bend.

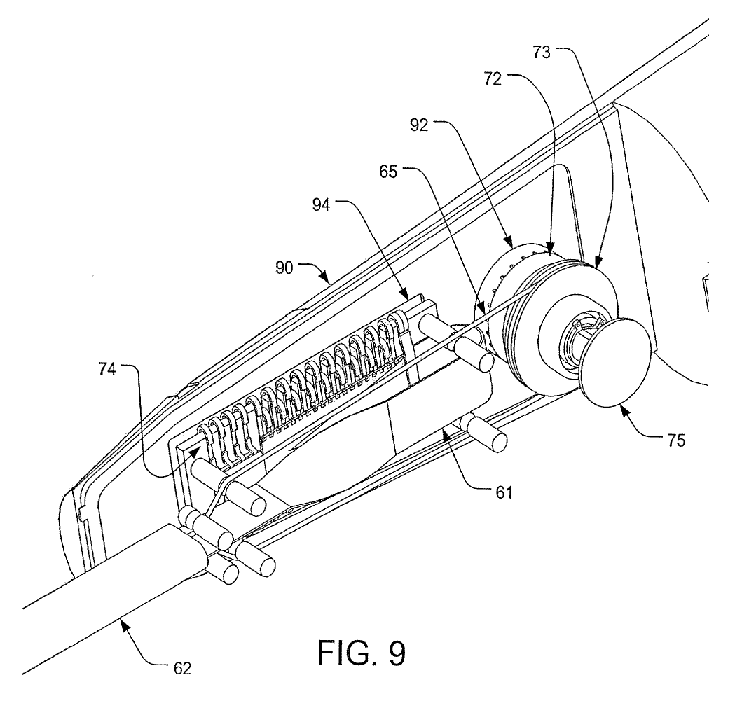

FIG. 9 shows the electrical and mechanical interactions between the first connector 90 and the second connector 70 when those connectors are mated together. This view depicts the mated set of connectors 90, 70 would look if the outside housing of the second connector 70 were invisible. The second electrical interface 74 is lined up with and urged against the first electrical interface 94, and the control actuator 72 on the second connector 70 is lined up with and urged against the output actuator 92 on the first connector 90. A pulley mount 75 permits the pulley 73 to rotate and urges the control actuator 72 against the output actuator 92 when the first connector 90 and second connector 70 are mated. The ribbon cable 61 that connects the second electrical interface 74 to the transducer 68 (shown in FIG. 2) at the distal end 66 of the transducer assembly 60 is also more clearly visible in this view.

When the second connector 70 is mated with the first connector 90, actuation of the user operated actuator 82 (shown in FIGS. 3 and 4) will cause the output actuator 92 to rotate. Since the control actuator 72 is being urged up against the output actuator 92, the control actuator 72 will follow the rotation of the output actuator 92. Rotation of the control actuator 72 turns the pulley 73 which operates the pull wires 65 that extend distally through the flexible shaft 62, and cause a bending mechanism (not shown) located in the bending section (shown in FIG. 3) to bend. Thus, when the second connector 70 is mated to the first connector 90, actuation of the user operated actuator 82 by the user will have the same net effect of actuations of the user operated actuator 102 of the unitary probe 100 depicted in FIG. 1. Note that while FIGS. 4-9 depict using rotating pads for the output actuator 92 and the control actuator 72 pads, numerous alternative mechanical interfaces (e.g., gears, a hexagonal shaft and a mating socket, etc.) may be substituted therefore, as will be appreciated by persons skilled in the relevant arts.

In addition, when the second connector 70 is mated with the first connector 90, the second electrical interface 74 makes contact with the first electrical connector 94. Since the first electrical connector 94 communicates with the ultrasound system 40 via cable 86 and connectors 88, 42 (all shown in FIG. 2), and Since the wiring 61 connects the second electrical interface 74 to the transducer 68 at the distal end 66 of the transducer assembly 60 (shown in FIGS. 2, 3) this arrangement permits the ultrasound system 40 to interface with the transducer 68 in the same way that the ultrasound system 40 communicates with the transducer 118 in the unitary probe 100 depicted in FIG. 1. Optionally, additional signals may be passed to and from the transducer assembly 60 via the first and second connectors 90, 70, e.g., to operate a thermistor located in the distal end of the transducer assembly 60 or to interface with a non-volatile memory device located in the transducer assembly 60 (used, e.g., to store data relating to the transducer assembly 60).

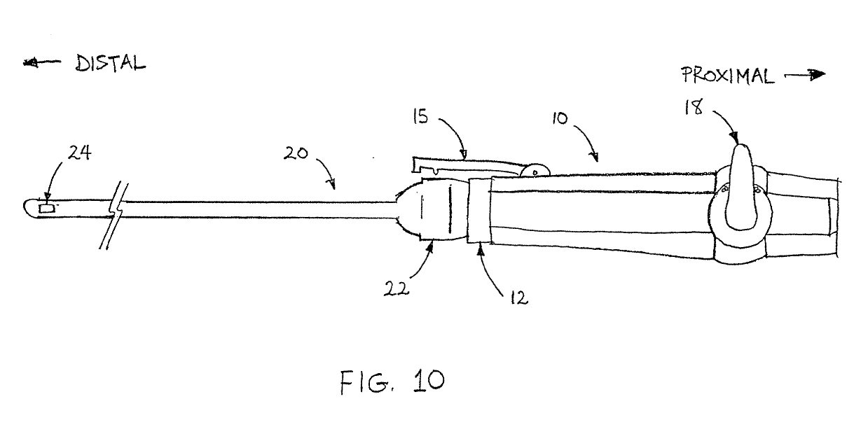

As best seen in FIGS. 4 and 9, the electrical and mechanical interface between the transducer assembly 60 and the actuator assembly 80 is sideways-facing (i.e., the mating surfaces of the first and second connectors 90, 70 face in a direction that is roughly perpendicular to the proximal-distal axis). This arrangement stands in contrast to the situation where one mating surface faces distally, and the other mating surface faces proximally (like the interface between the connectors 12, 22 in the FIG. 10 embodiment described below). Using a sideways-facing interface advantageously provides a large amount of “real estate” (i.e., area) for implementing the electrical and mechanical connections between the two assemblies. Moreover, despite the fact that a large amount of real estate is available for the interface, the overall diameter of the assemblies 60, 80 when connected can remain small (e.g., about 22 mm, measured at the proximal end of second connector 70 in the embodiment illustrated in FIGS. 3-9), and does not have to increase in proportion to the number of connections that are made between the first and second connectors 90, 70.

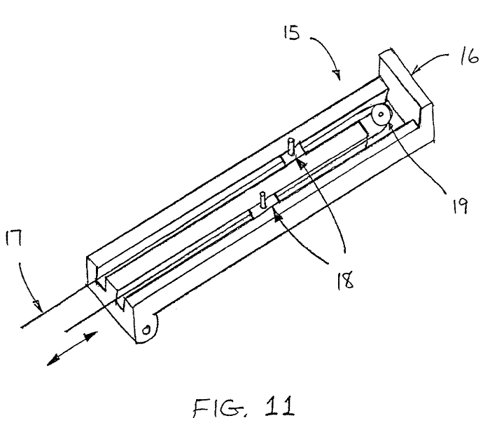

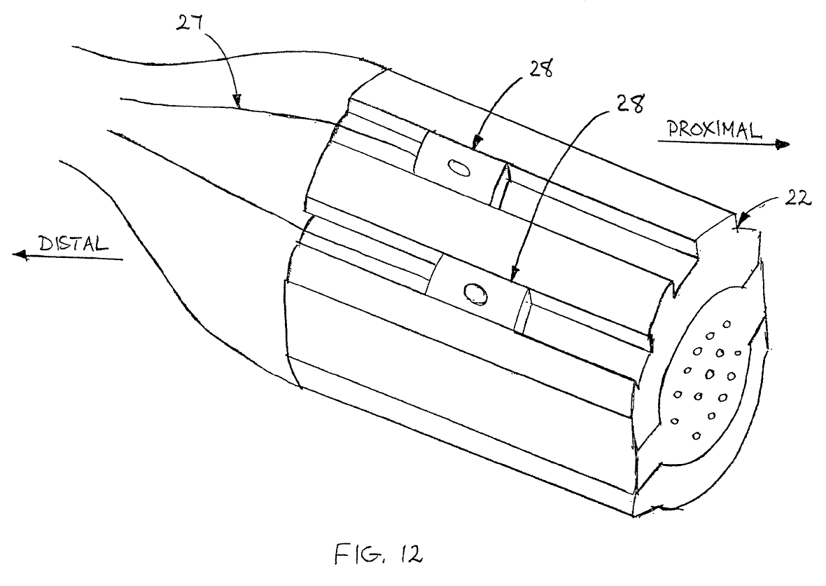

FIG. 10 is another embodiment of the invention in which the insertion tube and acoustic block assembly (referred to above as the transducer assembly) are separable from the control handle (referred to above as the actuator assembly). In this embodiment, a durable handle 10 is connected to the transducer assembly 20. A connector 12 at the distal end of the handle 10 mates with a corresponding connector 22 at the proximal end of the transducer assembly 20. FIG. 11 shows a detail of the latching arm 15 of the handle portion 10, and FIG. 12 shows a detail of the connector portion 22 of the transducer assembly 20.

Referring now to FIGS. 10-12, the connectors 12, 22 provide a detachable electrical interface to get all the necessary electrical signal to the distal end of the probe, and to receive return signals from the distal end of the probe. For example, the electrical connections may be used to pass signals used for generation of ultrasound at the ultrasound transducer 24, return of electrical signals from the transducer, ground and shielding planes, and any other electrical functions that are implemented at the distal end (e.g., connections to a non-volatile memory device may be integrated into the transducer assembly).

The connector 22 and the arm 15 also provide a detachable mechanical interface to actuate controllable portions at or near the distal end of the probe. An example of a desirable mechanical motion is flexing of the tip of the probe, which may be useful after the probe has been positioned in the fundus of the stomach. In the illustrated embodiment, the mechanical interface is implemented using pull wires that are connected to the distal end of the probe, where they initiate the desired motion (e.g., flexing of the probe tip). The mechanism that responds to the pull wires at the distal end of the probe may be implemented in any conventional manner. At the proximal end of the transducer assembly 20, the pull wires terminate in sliders 28 with a female hole.

To use the probe, the connector 22 is mated with the corresponding connector 12 of the handle, and the latching arm 15 is moved into position so that its pins 18 are mated into the sliders 28 of the transducer assembly 20. The latching arm may include a catch 16 to hold the transducer assembly 20 to the handle portion 10. The slides 18 are connected to each other via flexible cabling 17 which traverses a pulley 19 at the distal end of the latching arm 15. This configuration helps insure that articulation control cable stays taut within the handle and does not require the use of springs to take up slack.

The handle 10 includes a control surface 18 which may be implemented in any conventional way e.g., using pull wires. However, instead of having the pull wires go directly to the distal end of the probe, the pull wires the handle move the sliders 18 in the arm 15. Those sliders 18 in turn move the sliders 28, which move the pull wires 27 that run through the lumen of the transducer assembly 20 to generate the desired motion at the distal end of the probe. The result is a distal articulation mechanism that passes through a connector.

One suitable way to implement the electrical connection between the connectors 12, 22 is to use a flexible printed circuit board (PCB) similar to the type used in ink jet cartridge connectors. The reverse side of this flexible PCB has traces which are pulled out and connected to the appropriate cabling. Optionally, a chip with non-volatile memory may also be mounted on the flexible PCB. A suitable mating connector for this interface is a “pogo pin” type interface with pins mounted in a block (not shown), as commonly used in electronic testing apparatus.

Optionally, the actuator assembly in any of the embodiments described above may incorporate other actuatable features in addition to the basic articulation controls for manipulating the distal end of the insertion tube and transducer. For example, other mechanical connections besides the bending controls discussed above may be implemented, e.g., to transfer torque to the distal end of the probe. Controls for non-mechanical features may also be implemented on the handle, e.g., buttons for freezing the image, adjusting gain control or other functions. Optionally, the mechanical and electrical connections may be configured to be water-tight.

In all the above-described embodiments, when the transducer assembly is connected to the actuator assembly via the connector or connectors, the combination of the transducer assembly with the actuator assembly emulates both the electrical and mechanical operation of a conventional ultrasound probe. However, with the embodiments described above in connection with FIGS. 2-12, the doctor gains the ability to disconnect the actuator assembly from the transducer assembly, and leave the relatively compact distal transducer assembly section in position in the patient's esophagus. When this is done, only the connector 70, 22 and a portion of the flexible shaft 62, 20 will remain attached to the patient's body, and the handle, the actuator, and the cable that links the handle to the ultrasound system are disconnected from the patient. Since the hardware that stays attached to the patient's is smaller and lighter, it becomes much easier to move the patient around and to attend to the patient's needs, and is much less cumbersome as compared to the FIG. 1 embodiment in which the handle 104 and cable 106 stay attached to the patient as long as the transducer remains in position in the patient's esophagus. Preferably, the transducer assembly is configured so that the portion of the transducer assembly that remains outside of the patient's body is compact and has a mass of 250 g or less and a length of 70 cm or less.

Reducing the amount of hardware that is attached to the patient's is particularly advantageous for long term transesophageal ultrasound imaging, e.g., in situations where the probe remains installed in the patient for hours or days at a time. These advantages become even more important if the patient is awake or is not anesthetized, in which patient comfort becomes an even more important factor.

Advantages of the above-described embodiments include the fact that the device can be placed and left in-situ without causing problems with excessive bulk or cabling. In addition, by making the handle/actuator assembly separable from the transducer assembly, the transducer assembly may be made disposable and the handle may be made durable and reusable. This allows a less expensive disposable than would be possible if the entire probe were made disposable. It also allows the handle to be made to a higher standard than possible if the handle was also disposable, which may improve the tactile feedback to the user and ease of use.

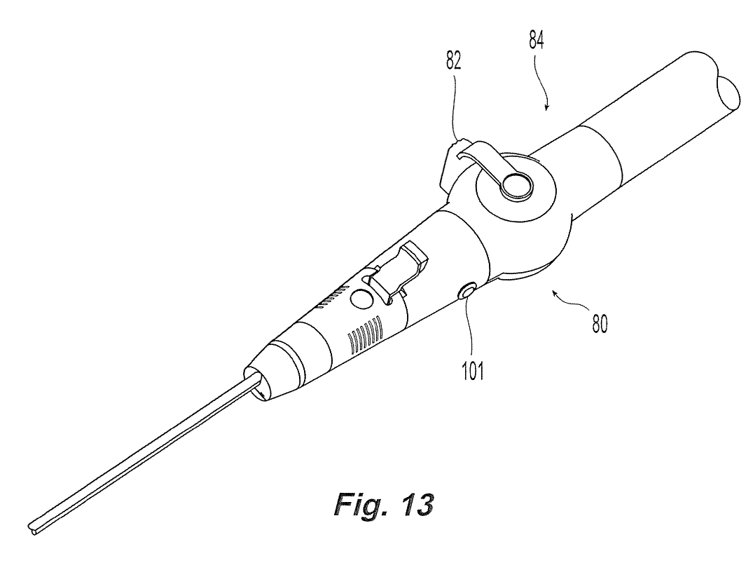

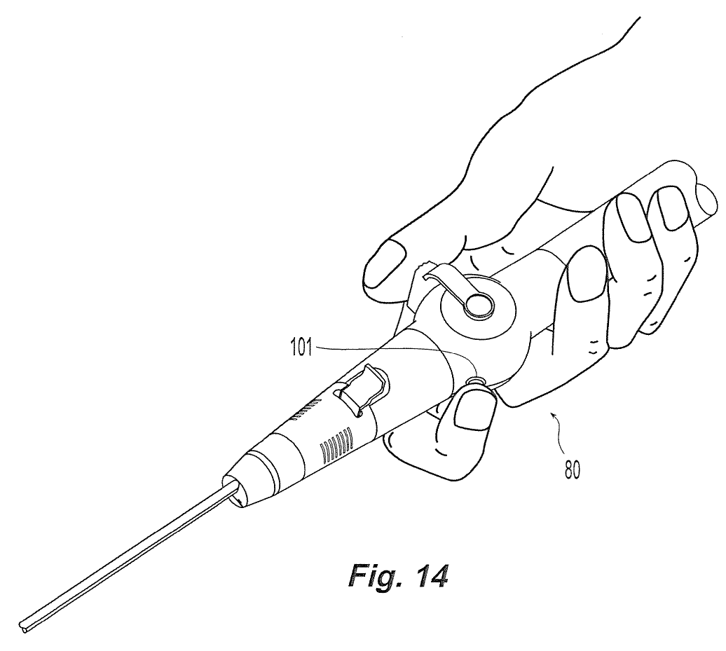

In additional preferred embodiments, illustrated in FIGS. 13 and 14, a preferred arrangement of controls of the embodiment of FIG. 3 is shown, with the description relating to FIG. 3 applying entirely to the embodiment illustrated in FIGS. 13 and 14 with identical features not identified again. As illustrated in FIGS. 13 and 14, the actuator assembly 80 includes the actuator 82 and the handle 84, and further includes a control button 101. The control button 101 is located on a longitudinal side of the handle 84 that is approximately opposite to a side of the handle 84 where an actuator 82 is located. The control button 101 can be positioned distal of the actuator 82, as illustrated in FIG. 13, or at a position along the longitudinal length of the actuator assembly 80 proximate to the actuator 82, as illustrated in FIG. 14. As illustrated in FIGS. 13 and 14, the control button 101 is disposed in a location on the actuator assembly 80 that is approximately opposite to the location of the actuator 82. By placing the control button 101 in a location approximately opposite to the location of the actuator 82, an ergonomic design is achieved that permits an operator grasping the handle 84 to simultaneously operate with a single hand the actuator 82 and the control button 101, as illustrated in FIG. 14. Furthermore, the control button 101 is positioned at a location that naturally corresponds to the forefinger, as illustrated in FIG. 14, when the operator grasps the handle 84. By this arrangement, when an operator grasps the handle 84 with a single hand, the thumb naturally rests near the actuator 82 and the forefinger naturally rests near the control button 101.

The control button 101 is preferably connected to wiring (not shown) internal to the handle 84. The wiring preferably connects to the ultrasound system 40 to activate a feature of the ultrasound system 40 with the activation of the control button 101. For example, the activation of the control button 101 can cause the ultrasound system 40 to take a still image of the moving images displayed by the monitor 44, or capture a loop of images or video to be displayed by the monitor 44. The control button 101 can also be made to control multiple functions of the ultrasound system 40, to cycle through several functions, and to select a function from a menu. The control button can also be made to operate in conjunction with another control device, such as a foot pedal, to allow the operator the ability to select and activate desired features of the ultrasound system 40.

Alternatively, the wiring can connect the control button 101 to equipment other than the ultrasound system 40, such as to another imaging system, medical device, or probe. For example, the control button 101 can control the operation or activation of recording device that captures an image provided by the ultrasound system 40.

In another alternative, the wiring can connect the control button 101 to a portion of the first electrical interface, so as to provide the connectorized connection described in regard to the probe illustrated in FIG. 3. For example, the control button 101 can be made to request an operation of the ultrasound transducer 68, or operate another transducer or sensor provided at the distal end 66 of the probe illustrated in FIG. 3.

While the above-described embodiments are discussed in the context of transesophageal echocardiography, similar probes may be used to obtain other transesophageal images as well as to obtain ultrasound images in cavities other than the esophagus, outside of the body when access is limited, or in non-medical applications. The connectorized construction may also be incorporated into probes, endoscopes, or catheters in non-ultrasound medical applications, and may even be used in non-medical uses where it is desirable to disconnect a proximal section while leaving the distal section in place. Numerous other modifications to the above-described embodiments will be apparent to persons skilled in the relevant arts, and are also included within the purview of the invention. For example, instead of an ultrasound transducer 68, the connectorized construction can be used with an optical probe, with a sensor that is acoustic, electrical, or magnetic, or with a device that emits or detects radiation or vibrations.

We claim:

1. A handle for an ultrasound probe that has a bendable distal portion, comprising:
a gripping portion configured to be comfortably held in a user's hand while the handle is being used;
a control region positioned distal to the gripping portion, the control region having a top and a bottom;
a first actuator disposed on the top of the control region, and positioned such that when the gripping portion is being held in the user's hand, the first actuator is located just beneath a natural resting place of the user's thumb, wherein the first actuator can be moved back and forth in a proximal-distal direction by the user's thumb, and wherein the first actuator is configured so that (a) actuation of the first actuator causes the distal portion of the ultrasound probe to bend and (b) deactuation of the first actuator causes the distal portion of the probe to unbend; and
a second actuator disposed on the bottom of the control region, and positioned such that when the gripping portion is being held in the user's hand with the first actuator located beneath the user's thumb, the second actuator is located adjacent to a natural resting place of the user's forefinger so that the second actuator can be easily actuated by the user's forefinger, wherein actuation of the second actuator generates a signal that is compatible with a control input of an ultrasound system.

2. The handle of claim 1, wherein the second actuator comprises a pushbutton switch.

3. The handle of claim 2, wherein the signal initiates capturing of an image.

4. The handle of claim 1, wherein the first actuator comprises a lever.

5. The handle of claim 1, wherein the first actuator comprises a lever and the second actuator comprises a pushbutton switch.

6. The handle of claim 5, wherein the signal initiates capturing of an image.

7. The handle of claim 1, wherein the handle attaches to the bendable distal portion with a connectorized connection that provides control over bending of the bendable distal portion.

8. The handle of claim 1, wherein the handle attaches to the bendable distal portion with a non-connectorized connection.

9. A handle for an ultrasound probe that has a bendable distal portion, the handle comprising:
a body;
a first actuator located on a first side of the body and positioned so as to be easily actuatable by a user's thumb when the body is being held in the user's hand, wherein the first actuator is configured so that (a) actuation of the first actuator causes the distal portion of the ultrasound probe to bend and (b) deactuation of the first actuator causes the distal portion of the probe to unbend; and
a second actuator located on a side of the body that is opposite to the first side of the body and positioned so as to be easily actuatable by the user's forefinger when the body is being held in the user's hand and the user's thumb is positioned on the first actuator, wherein actuation of the second actuator generates a signal that is compatible with a control input of an ultrasound system.

10. The handle of claim 9, wherein the second actuator comprises a pushbutton switch.

11. The handle of claim 10, wherein the signal initiates capturing of an image.

12. The handle of claim 9, wherein the first actuator comprises a lever.

13. The handle of claim 9, wherein the first actuator comprises a lever and the second actuator comprises a pushbutton switch.

14. The handle of claim 13, wherein the signal initiates capturing of an image.

15. The handle of claim 9, wherein the handle attaches to the bendable distal portion with a connectorized connection that provides control over bending of the bendable distal portion.

16. The handle of claim 9, wherein the handle attaches to the bendable distal portion with a non-connectorized connection.
